(19) United States
(12) Patent Application Publication
(10) Pub. No.: US 2021/0052486 A1
(43) Pub. Date: Feb. 25, 2021

(54) SKIN-WHITENING COMPOSITION WITH FERMENTATION METABOLITE OF LACTIC ACID BACTERIUM AND APPLICATIONS THEREOF

(71) Applicant: GLAC BIOTECH CO., LTD., TAINAN CITY (TW)

(72) Inventors: PEI-SHAN HSIEH, Tainan City (TW); CHUNG-WEI KUO, Tainan City (TW); YI-CHUN TSAI, Tainan City (TW); HSIEH-HSUN HO, Tainan City (TW); YI WEI KUO, Tainan City (TW); JIA-HUNG LIN, Tainan City (TW)

(21) Appl. No.: 16/939,936

(22) Filed: Jul. 27, 2020

(30) Foreign Application Priority Data
Aug. 22, 2019 (CN) 201910778700.6

(51) Int. Cl. A61K 8/99; A61Q 19/02

(52) U.S. Cl. CPC A61K 8/99; A61Q 19/02

(57) ABSTRACT

A fermentation metabolite generated by an isolated lactic acid bacterium including at least one of a Bv-889 strain of Bifidobacterium breve, a BLI-02 strain of Bifidobacterium longum subsp. infantis, a CP-9 strain of Bifidobacterium animalis subsp. lactis, a Bf-688 strain of Bifidobacterium bifidum, an AP-32 strain of Lactobacillus salivarius subsp. salicinius, and a GL-156 strain of Lactobacillus paracasei. The fermentation metabolite of the abovementioned strains have a skin-whitening effect and is in form of a food composition or a cosmeceutical composition.

### BACKGROUND OF THE INVENTION

### 1. Field of the Invention

The present invention relates to a composition and applications thereof, particularly to a skin-whitening composition with a fermentation metabolite of a lactic acid bacterium and application thereof.

### 2. Description of the Prior Art

Beauty is human nature. White and bright skin is a dream many oriental women are eager to realize. Long-term exposure to pollution, carcinogen and ultraviolet light is very likely to cause damage to skin. Melanocytes may generate melanin to decrease the damage to skin. Melanin can absorb most of ultraviolet light, protecting skin and allaying acute and chronical damage induced by ultraviolet light.

Melanin accumulates in skin, and cellular metabolism of skin will eliminate melanin in the basal layer, wherein cellular metabolism replaces the cells where melanin accumulates with new cells. Thus, the sunlight-exposed skin will be restored to its original color. As some intrinsic and extrinsic factors, such as aging, sleep deficit, ultraviolet light and life pressure, persistently affect skin, the rate of melanin formation will finally overrun the rate of cellular metabolism, which accelerates skin aging, darkens skin, causes freckles, or even brings about skin cancers.

Accordingly, it is an urgency to develop a nutrient or cosmetic product, which can be used long term and is able to whiten skin. In general, fermentation metabolites of lactic acid bacteria are safe and healthy for human beings. Therefore, finding out fermentation metabolites of lactic acid bacteria able to whiten skin becomes a target the manufacturers are eager to achieve.

### SUMMARY OF THE INVENTION

The present invention provides a skin-whitening composition with a fermentation metabolite of a lactic acid bacterium, which has an active effect of inhibiting generation of melanin, whereby the present invention can reduce accumulation of melanin, whiten skin and improve skin quality.

In one embodiment, the skin-whitening composition with a fermentation metabolite of a lactic acid bacterium of the present invention comprises a fermentation metabolite generated by an isolated lactic acid bacterium strain and having a skin-whitening effect, and an excipient, diluent or carrier. The lactic acid bacterium strain comprises at least one of a Bv-889 strain of Bifidobacterium breve (CGMCC No. 16145); a BLI-02 strain of Bifidobacterium longum subsp. infantis (CGMCC No. 15212); a CP-9 strain of Bifidobacterium animalis subsp. lactis (CCTCC NO: M2014588); a Bf-688 strain of Bifidobacterium bifidum (CGMCC No. 17953); an AP-32 strain of Lactobacillus salivarius subsp. salicinius (CCTCC NO: M2011127); and a GL-156 strain of Lactobacillus paracasei (CCTCC NO: M2014590).

In another embodiment, the present invention proposes a use of a composition with a fermentation metabolite of a lactic acid bacterium for skin whitening comprising administering to a subject the composition, wherein the composition with a fermentation metabolite of a lactic acid bacterium comprises a fermentation metabolite generated by an isolated lactic acid bacterium strain and having a skin-whitening effect, and an excipient, diluent or carrier. The lactic acid bacterium strain comprises at least one of a Bv-889 strain of Bifidobacterium breve (CGMCC No. 16145), a BLI-02 strain of Bifidobacterium longum subsp. infantis (CGMCC No. 15212), a CP-9 strain of Bifidobacterium animalis subsp. lactis (CCTCC NO: M2014588), a Bf-688 strain of Bifidobacterium bifidum (CGMCC No. 17953), an AP-32 strain of Lactobacillus salivarius subsp. salicinius (CCTCC NO: M2011127), and a GL-156 strain of Lactobacillus paracasei (CCTCC NO: M2014590). The abovementioned strains are respectively deposited in China General Microbiological Culture Collection Center (CGMCC) and China Center for Type Culture Collection (CCTCC).

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 shows the results of the experiments to evaluate the melanin-inhibiting capabilities of the fermentation metabolites of the lactic acid bacterium strains of the present invention; and

FIG. 2 shows the results of the experiments to evaluate the effects of inhibiting the tyrosinase activity of the fermentation metabolites of lactic acid bacteria of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The freeze-dried cultures of the lactic acid bacterium strains mentioned in the specification are deposited in China General Microbiological Culture Collection Center (CGMCC) of Chinese Academy of Sciences (NO. 1 West Beichen Road, Chaoyang District, Beijing 100101, China)) and China Center for Type Culture Collection (CCTCC) of Wuhan University (Wuhan 430072, China). The details thereof are listed in Table. 1.

TABLE 1
Data of Deposited Lactic Acid Bacterium strains

| | | Deposition | Deposition |
|---|---|---|---|
| Strain | Specie | No. | Date |
| Bv-889 | Bifidobacterium breve | CGMCC No. | Jul. 23, |
| | | 16145 | 2018 |
| BLI-02 | Bifidobacterium longum | CGMCC No. | Jan. 15, |
| | subsp. infantis | 15212 | 2018 |
| CP-9 | Bifidobacterium animalis | CCTCC NO: | Nov. 24, |
| | subsp. lactis | M2014588 | 2014 |
| Bf-688 | Bifidobacterium bifidum | CGMCC No. | Jun. 18, |
| | | 17953 | 2019 |
| Ap-32 | Lactobacillus salivarius | CCTCC NO: | Apr. 10, |
| | subsp. salicinius | M2011127 | 2011 |
| GL-156 | Lactobacillus paracasei | CCTCC NO: | Nov. 24, |
| | | M2014590 | 2014 |

It is found: the fermentation metabolites of the deposited strains listed in Table. 1 have the effects of inhibiting melanin formation and inhibiting melanin synthase, including the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, the GL-156 strain of Lactobacillus paracasei. It is easily understood: the effect of inhibiting melanin formation is equal to the effect of whitening skin and fading freckles; the effect of inhibiting melanin synthase is equal to the effect of inhibiting melanin formation. Therefore, in order to simplify the description, the effect of skin whitening is used to represent the effect of inhibiting melanin formation and the effect of inhibiting melanin synthase in the specification unless it is noted specially.

In one embodiment, the fermentation metabolites of the lactic acid bacterium strains listed in Table. 1 may be used in skin whitening. In one embodiment, the skin-whitening composition with a fermentation metabolite of a lactic acid bacterium of the present invention comprises a fermentation metabolite generated by a lactic acid bacterium strain, and an excipient, diluent or carrier. The lactic acid bacterium strain is at least one isolated lactic acid bacterium strain selected a group including a Bv-889 strain of Bifidobacterium breve (CGMCC No. 16145), a BLI-02 strain of Bifidobacterium longum subsp. infantis (CGMCC No. 15212), a CP-9 strain of Bifidobacterium animalis subsp. lactis (CCTCC NO: M2014588), a Bf-688 strain of Bifidobacterium bifidum (CGMCC No. 17953), an AP-32 strain of Lactobacillus salivarius subsp. salicinius (CCTCC NO: M2011127), and a GL-156 strain of Lactobacillus paracasei (CCTCC NO: M2014590). The abovementioned strains are respectively deposited in China

General Microbiological Culture Collection Center (CGMCC) and China Center for Type Culture Collection (CCTCC). In one embodiment, the excipient, diluent or carrier is a physiologically-acceptable excipient, diluent or carrier; thus, the skin-whitening composition with a fermentation metabolite of a lactic acid bacterium of the present invention may be used as a food composition. In one embodiment, the excipient, diluent or carrier is a cosmeceutically-acceptable excipient, diluent or carrier; thus, the skin-whitening composition with a fermentation metabolite of a lactic acid bacterium of the present invention may be used as a cosmeceutical composition.

In the embodiment of the food composition, the physiologically-acceptable excipient, diluent or carrier may be a food. The food may be but is not limited to be dairy food, tea, coffee, a chewing gum, a tooth-cleaning candy (such as an oral strip, a chewable tablet, or jelly sweets), or a combination thereof. The dairy food may be fermented milk, yoghurt, cheese, milk drink, or powdered milk.

In the embodiment of the cosmeceutical composition, the cosmeceutically-acceptable excipient, diluent or carrier may be 1) a liquid cosmetic, such as a shower gel, a shampoo, a lotion, or a perfume; 2) an emulsion type cosmetic; 3) a cream type cosmetic, such as a facial cream, a foundation cream, or a shampoo paste; 4) a powdered cosmetic, such as a fragrance powder, or a talcum powder; 5) a block type cosmetic, such as a pressed powder, or a cosmetic box; 6) a stick type cosmetic, such as a lipstick, or a hair wax.

In one embodiment, the fermentation metabolite generated by the lactic acid bacterium strain of the present invention may contain a inactivated strain, a fermentation liquid where bacteria are removed, or a dried powder of a fermentation liquid where bacteria are removed. In one embodiment, the fermentation liquid may be a supernatant of fermentation, or a fermentation whey. In one embodiment, the skin-whitening composition with a fermentation metabolite of a lactic acid bacterium contains more than 0.5% of the dried powder of the fermentation metabolite or more than 2.5% of the fermentation liquid of the fermentation metabolite.

### Embodiment I: Morphology and General Properties of the Strains of the Present Invention

The taxonomic characteristics of the strain are identified with the 16S rDNA sequencing analysis and the API bacterial identification system. The morphology and general properties of the strains are listed in Table. 2.

| Strain | Morphology and characteristics |
|---|---|
| Bv-889 of | 1. They are gram-positive bacilli, unlikely to |
| Bifidobacterium | generate spores, free of catalase, oxidase and |
| breve | motility, able to grow in obligately-anaerobic |
| | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bacterium |
| | body has a middle-size or shorter rod-like shape, |
| | and two ends thereof sometimes have Y or |
| | V-shaped branches. |
| BLI-02 of | 1. They are gram-positive bacilli, unlikely to |
| Bifidobacterium | generate spores, free of catalase, oxidase and |
| longum | motility, able to grow in obligately-anaerobic |
| subsp. infantis | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bacterium |
| | body has a middle-size or longer rod-like shape, |
| | and two ends thereof sometimes have Y or |
| | V-shaped branches. |
| CP-9 of | 1. They are gram-positive bacilli, unlikely to |
| Bifidobacterium | generate spores, free of catalase, oxidase and |
| animalis | motility, able to grow in obligately-anaerobic |
| subsp. lactis | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bacterium |
| | body has a middle-size or longer rod-like shape, |
| | and two ends thereof sometimes have Y or |
| | V-shaped branches. |
| Bf-688 of | 1. They are gram-positive bacilli, unlikely to |
| Bifidobacterium | generate spores, free of catalase, oxidase and |
| bifidum | motility, able to grow in obligately-anaerobic |
| | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bacterium |
| | body has a middle-size or longer rod-like shape, |
| | and two ends thereof sometimes have Y or |
| | V-shaped branches. |
| AP-32 of | 1. They are gram-positive bacilli, unlikely to |
| Lactobacillus | generate spores, free of catalase, oxidase and |
| salivarius | motility, able to grow in aerobic and anaerobic |
| subsp. salicinius | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bodies |
| | of the bacteria each have a shape of a short |
| | rod, and the ends of the body are circular- |
| | shaped. They often appear in single bodies. |
| GL-156 of | 1. They are gram-positive bacilli, unlikely to |
| Lactobacillus | generate spores, free of catalase, oxidase and |
| paracasei | motility, able to grow in aerobic and anaerobic |
| | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bodies |
| | of the bacteria each have a shape of a short |
| | rod, and the ends of the body are circular- |
| | shaped. They often appear in single bodies. |

### Embodiment II: Collection and Preservation of the Fermentation Metabolites of Lactic Acid Bacterium Strains

The fermentation metabolite of the present invention is the fermentation product generated by at least one of the lactic acid bacterium strains: the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei. The fermentation product is centrifuged, filtered, sterilized and then purified to obtain a fermentation liquid. According to requirement, the fermentation liquid is further dried to form fermentation powder of the lactic acid bacterium. The fermentation liquid or fermentation powder can be stored at an ambient temperature. The Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum are separated from human breast milk. The AP-32 strain of Lactobacillus salivarius subsp. Salicinius is separated from human excrement. The GL-156 strain of Lactobacillus paracasei is separated from human intestines.

### Embodiment III: Analysis of the Melanin-Inhibition Capability of the Fermentation Metabolites of Lactic Acid Bacteria

Melanoma cells are widely used to analyze cell models of melanoma formation. In the experiment, ultraviolet illumination is used to induce formation of melanin. Melanoma cells may be activated to generate melanin by melanocyte-stimulating hormones (α-MSH). The generated melanin may be quantitatively measured via measuring OD_{400 nm}. In brief, the cells are dissolved to extract melanin; a spectrophotometer is used to measure the absorption of OD₄₀₀ₘₙ; the higher the absorption, the more the melanin generated by melanoma cells.

In the experiment, mix 100 nM α-MSH with 2.5% and 7.5% aqueous solution of the fermentation powder of the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei to form a first solution. Next, co-culture the first solution and melanoma cells (B16-F10) for 48 hours. As arbutin can inhibit melanin formation, 1 mM and 2 mM arbutin is mixed with the melanoma cells having been mixed with a-MSH to function as a positive control group. The melanoma cells that are only stimulated by a-MSH is used as the negative control group. After the cells are dissolved, the amount of the melanin generated by each group is obtained via measuring OD_{400 nm}.

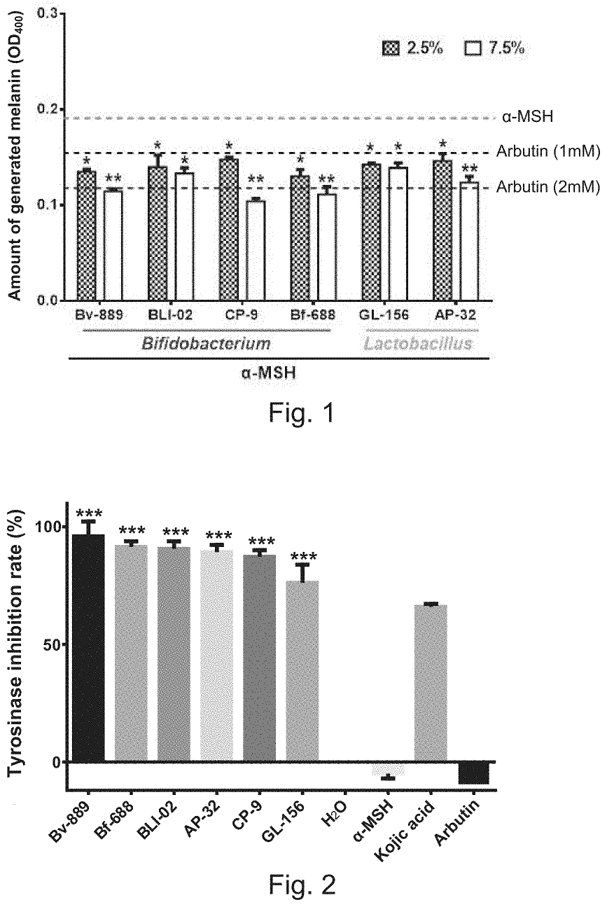

Refer to FIG. 1 for the results of the experiments to evaluate the melanin-inhibiting capabilities of the fermentation metabolites of the lactic acid bacterium strains of the present invention, wherein ** denotes p<0.01, indicating a high degree of statistical significance; * denotes p<0.05, indicating statistical significance. In FIG. 1, the topmost dashed line denotes the negative control group; the lower two dashed lines denote the positive control groups. From FIG. 1, it is learned: the fermentation metabolites of the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei of the present invention can effectively inhibit the melanin formation induced by a-MSH. The effects of the fermentation metabolites of the present invention are equal to the effects of arbutin. Therefore, the fermentation metabolites generated by the lactic acid bacterium strains of the present invention has a skin-whitening effect.

### Embodiment IV: Analysis of the Effect of Inhibiting the Tyrosinase Activity of the Fermentation Metabolites of Lactic Acid Bacteria

Tyrosinase plays an important role in the synthesis of melanin. Tyrosinase is an oxidase, which is a rate-limiting enzyme for regulating melanin formation. Tyrosinase take part in two reactions in melanin synthesis: the first reaction hydroxylates monophenol into diphenol; the second reaction oxidizes an o-diphenol into an o-quinone. o-Quinone undergoes several reactions to eventually form melanin. The higher the activity of tyrosinase, the more the generated melanin. The amount of the generated melanin is proportional to the activity of tyrosinase. Therefore, inhibiting the activity of tyrosinase can reduce the amount of the generated melanin. For example, Kojic acids may be used to inhibit the activity of tyrosinase and achieve the skin-whitening effect. The fermentation powders of the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei of the present invention are prepared to form 20% aqueous solutions to analyze the effect of inhibiting the activity of tyrosinase. Water is used as a blank experiment group. A 0.75 nM aqueous solution of Kojic acid and a 2 nM aqueous solution of arbutin are used as positive control groups. A 100 nM α-MSH solution is used as a negative control group. A Tyrosinase Inhibitor Screening Kit (BioVision) is used in the experiment to analyze the effect of inhibiting the tyrosinase activity; all the experimental processes are performed according to the proposals in the manual of the abovementioned kit.

Refer to FIG. 2 for results of the experiments to evaluate the effects of inhibiting the tyrosinase activity of the fermentation metabolites of lactic acid bacteria of the present invention, wherein *** denotes p<0.005, indicating a high degree of statistical significance. It is learned from FIG. 2: in comparison with the negative control group (the group using a-MSH) and the blank control group, the fermentation metabolites of the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei of the present invention has an effect of inhibiting the tyrosinase activity. The effect of inhibiting the tyrosinase activity of the present invention is parallel or even superior to the effect of Kojic acids and arbutin, which are known for their skin-whitening effect.

In conclusion, the fermentation metabolites generated by the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei of the present invention have the capability of inhibiting melanin formation and the capability of inhibiting melanin synthase. Therefore, the fermentation metabolites generated by the Bv-889 strain of Bifidobacterium breve, the BLI-02 strain of Bifidobacterium longum subsp. infantis, the CP-9 strain of Bifidobacterium animalis subsp. lactis, the Bf-688 strain of Bifidobacterium bifidum, the AP-32 strain of Lactobacillus salivarius subsp. salicinius, and the GL-156 strain of Lactobacillus paracasei of the present invention can whiten skin, fade freckles, and prevent from melanin formation.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

### Deposition of Biological Material for Patent Purposes

1. The Bv-889 strain of the present invention
Deposition Date: Jul. 23, 2018
Deposition Authority: China General Microbiological Culture Collection Center (CGMCC)
Address of Deposition Authority: Institute of Microbiology, Chinese Academy of Sciences, NO. 1 West Beichen Road, Chaoyang District, Beijing 100101, China
Deposition Number: CGMCC No. 16145
Taxonomic Name: Bifidobacterium breve
   2. The BLI-02 strain of the present invention
Deposition Date: Jan. 15, 2018
Deposition Authority: China General Microbiological Culture Collection Center (CGMCC)
Address of Deposition Authority: Institute of Microbiology, Chinese Academy of Sciences, NO. 1 West Beichen Road, Chaoyang District, Beijing 100101, China
Deposition Number: CGMCC No. 15212
Taxonomic Name: Bifidobacterium longum subsp. infantis
   3. The CP-9 strain of the present invention
Deposition Date: Nov. 24, 2014
Deposition Authority: China Center for Type Culture Collection (CCTCC)
Address of Deposition Authority: Wuhan University, Wuhan 430072, China
Deposition Number: CCTCC NO: M2014588
Taxonomic Name: Bifidobacterium animalis subsp. lactis
   4. The Bf-688 strain of the present invention
Deposition Date: Jun. 18, 2019
Deposition Authority: China General Microbiological Culture Collection Center (CGMCC)
Address of Deposition Authority: Institute of Microbiology, Chinese Academy of Sciences, NO. 1 West Beichen Road, Chaoyang District, Beijing 100101, China
Deposition Number: CGMCC No. 17953
Taxonomic Name: Bifidobacterium bifidum
   5. The AP-32 strain of the present invention
Deposition Date: Apr. 10, 2011
Deposition Authority: China Center for Type Culture Collection (CCTCC)
Address of Deposition Authority: Wuhan University, Wuhan 430072, China
Deposition Number: CCTCC NO: M2011127
Taxonomic Name: Lactobacillus salivarius subsp. salicinius
   6. The GL-156 strain of the present invention
Deposition Date: Nov. 24, 2014
Deposition Authority: China Center for Type Culture Collection (CCTCC)
Address of Deposition Authority: Wuhan University, Wuhan 430072, China
Deposition Number: CCTCC NO: M2014590
Taxonomic Name: Lactobacillus paracasei

What is claimed is:

1. A skin-whitening composition with a fermentation metabolite of a lactic acid bacterium, comprising:
a fermentation metabolite generated by an isolated lactic acid bacterium strain and having a skin-whitening effect, wherein the lactic acid bacterium strain comprises at least one of a Bv-889 strain of Bifidobacterium breve with a deposition number of CGMCC No. 16145, a BLI-02 strain of Bifidobacterium longum subsp. infantis with a deposition number of CGMCC No. 15212, a CP-9 strain of Bifidobacterium animalis subsp. lactis with a deposition number of CCTCC NO: M2014588, a Bf-688 strain of Bifidobacterium bifidum with a deposition number of CGMCC No. 17953, an AP-32 strain of Lactobacillus salivarius subsp. salicinius with a deposition number of CCTCC NO: M2011127, and a GL-156 strain of Lactobacillus paracasei with a deposition number of CCTCC NO: M2014590, wherein the abovementioned strains are respectively deposited in China General Microbiological Culture Collection Center (CGMCC) and China Center for Type Culture Collection (CCTCC); and
an excipient, diluent or carrier.

2. The skin-whitening composition with a fermentation metabolite of a lactic acid bacterium according to claim 1, wherein the fermentation metabolite comprises inactivated strains, or a supernatant of a fermentate liquid, a fermentate whey or a dried powder thereof in which bacteria are removed.

3. The skin-whitening composition with a fermentation metabolite of a lactic acid bacterium according to claim 1, wherein the excipient, diluent or carrier is a physiologically-acceptable excipient, diluent or carrier.

4. The skin-whitening composition with a fermentation metabolite of a lactic acid bacterium according to claim 1, wherein the excipient, diluent or carrier is a food.

5. The skin-whitening composition with a fermentation metabolite of a lactic acid bacterium according to claim 4, wherein the food is fermented milk, yoghurt, cheese, milk drink, powdered milk, tea, coffee, a chewing gum, a tooth-cleaning candy, or a combination thereof.

6. The skin-whitening composition with a fermentation metabolite of a lactic acid bacterium according to claim 1, wherein the excipient, diluent or carrier is a cosmeceutically-acceptable excipient, diluent or carrier.

7. The skin-whitening composition with a fermentation metabolite of a lactic acid bacterium according to claim 1, which is in form of a liquid cosmetic, an emulsion type cosmetic, a cream type cosmetic, a powdered cosmetic, a block type cosmetic, or a stick type cosmetic.

8. The skin-whitening composition with a fermentation metabolite of a lactic acid bacterium according to claim 1, which is in form of a shower gel, a shampoo, a lotion, a perfume, a facial cream, a foundation cream, a shampoo paste, a fragrance powder, a talcum powder, a pressed powder, a cosmetic box, a lipstick, or a hair wax.

9. A use of a composition with a fermentation metabolite of a lactic acid bacterium for skin whitening comprising administering to a subject the composition, wherein the composition with a fermentation metabolite of a lactic acid bacterium comprises:
a fermentation metabolite generated by an isolated lactic acid bacterium strain and having a skin-whitening effect, wherein the lactic acid bacterium strain comprises at least one of a Bv-889 strain of Bifidobacterium breve with a deposition number of CGMCC No. 16145, a BLI-02 strain of Bifidobacterium longum subsp. infantis with a deposition number of CGMCC No. 15212, a CP-9 strain of Bifidobacterium animalis subsp. lactis with a deposition number of CCTCC NO: M2014588, a Bf-688 strain of Bifidobacterium bifidum with a deposition number of CGMCC No. 17953, an AP-32 strain of Lactobacillus salivarius subsp. salicinius with a deposition number of CCTCC NO: M2011127, and a GL-156 strain of Lactobacillus paracasei with a deposition number of CCTCC NO: M2014590, wherein the abovementioned strains are respectively deposited in China General Microbiological Culture Collection Center (CGMCC) and China Center for Type Culture Collection (CCTCC); and
an excipient, diluent or carrier.

10. The use of a composition with a fermentation metabolite of a lactic acid bacterium for skin whitening according to claim 9, wherein the fermentation metabolite comprises inactivated strains, or a supernatant of a fermentate liquid, a fermentate whey or a dried powder thereof in which bacteria are removed.

11. The use of a composition with a fermentation metabolite of a lactic acid bacterium for skin whitening according to claim 9, wherein the excipient, diluent or carrier is a physiologically-acceptable excipient, diluent or carrier.

12. The use of a composition with a fermentation metabolite of a lactic acid bacterium for skin whitening according to claim 9, wherein the excipient, diluent or carrier is a food.

13. The use of a composition with a fermentation metabolite of a lactic acid bacterium for skin whitening according to claim 12, wherein the food is fermented milk, yoghurt, cheese, milk drink, powdered milk, tea, coffee, a chewing gum, a tooth-cleaning candy, or a combination thereof.

14. The use of a composition with a fermentation metabolite of a lactic acid bacterium for skin whitening according to claim 9, wherein the excipient, diluent or carrier is a cosmeceutically-acceptable excipient, diluent or carrier.

15. The use of a composition with a fermentation metabolite of a lactic acid bacterium for skin whitening according to claim 9, wherein the composition with a fermentation metabolite of a lactic acid bacterium is in form of a liquid cosmetic, an emulsion type cosmetic, a cream type cosmetic, a powdered cosmetic, a block type cosmetic, or a stick type cosmetic.

16. The use of a composition with a fermentation metabolite of a lactic acid bacterium for skin whitening according to claim 9, wherein the composition with a fermentation metabolite of a lactic acid bacterium is in form of a shower gel, a shampoo, a lotion, a perfume, a facial cream, a foundation cream, a shampoo paste, a fragrance powder, a talcum powder, a pressed powder, a cosmetic box, a lipstick, or a hair wax.
